(19) United States
(12) Patent Application Publication
(10) Pub. No.: US 2024/0033213 A1
(43) Pub. Date: Feb. 1, 2024

(54) TOPICAL PHARMACEUTICAL FORMULATION AND METHOD FOR THE TREATMENT OF SYNDROMES ASSOCIATED WITH CHRONIC PELVIC PAIN

(71) Applicant: CURALEAF INTERNATIONAL LIMITED, London (GB)

(72) Inventors: Barbara PACCHETTI, Locarno (CH); Antonio COSTANZO, London (GB)

(73) Assignee: CURALEAF INTERNATIONAL LIMITED, London (GB)

(21) Appl. No.: 18/257,796

(22) PCT Filed: Dec. 16, 2021
(86) PCT No.: PCT/EP2021/086077

§ 371 Date: Jun. 15, 2023

(30) Foreign Application Priority Data
Dec. 18, 2020 (IT) 102020000031379

(51) Int. Cl. A61K 9/00; A61K 31/00; A61K 9/127; A61K 9/06; A61P 25/02; A61K 47/14; A61K 47/36; A61K 47/38

(52) U.S. Cl. CPC A61K 9/0014; A61K 9/0009; A61K 31/658; A61K 9/1272; A61K 9/06; A61P 25/02; A61K 47/14; A61K 47/36; A61K 47/38

(57) ABSTRACT

A topical pharmaceutical formulation comprising an aqueous composition comprising cannabinoids, in particular cannabidiol, in niosomes having a size of less than 500 nm, and at least one topically acceptable excipient, for use in the treatment of syndromes associated with chronic pelvic pain, in particular by topical application by means of a dynamic electroporation apparatus.

### FIELD OF THE INVENTION

The present invention relates to a composition comprising cannabinoids for use in the treatment of syndromes associated with chronic pelvic pain.

In particular, the present invention relates to a pharmaceutical formulation for topical application comprising cannabidiol incorporated in niosomes, preferably having a size of less than 500 nm, and at least one topically acceptable excipient for use in the treatment of syndromes associated with chronic pelvic pain, in particular by means of a dynamic electroporation apparatus.

The present invention further relates to a method for treating syndromes associated with chronic pelvic pain by topical application of a pharmaceutical formulation comprising cannabinoids by means of a dynamic electroporation apparatus.

### STATE OF THE ART

Chronic pelvic pain (CPD) is defined as continuous or intermittent pain located in the hypogastrium (lesser pelvis) that persists for more than 6 months. CVD is a common condition that affects approximately one in every 6 women. Increased pain deteriorates patients' quality of life and requires pharmacological treatment or physical intervention. The etiology of DPC has not been fully defined and elucidated and the causes are usually complex.

DPC is persistent or recurrent episodic pelvic pain associated with symptoms suggesting lower urinary tract, sexual, bowel, or gynecologic dysfunction. Many patients with DPC present with complex manifestations, including pain and dysfunction that are not confined to a single organ system (lower urinary tract, lower gastrointestinal tract, genital tract). These syndromes may be associated with varying degrees of symptoms suggestive of bladder pain syndrome, dyspareunia (vulvodynia), and/or irritable bowel syndrome. These patients experience pain that includes a neuropathic component (burning, tingling, stinging, and perineal allodynia), and physical examination may reveal points of muscle activation (piriformis, obturator internus, levator ani, and iliopsoas) suggestive of myofascial pain. The underlying mechanism of this condition may be explained by repeated or prolonged somatic and visceral sensory impulses from nociceptors, resulting in a lowering of their activation threshold, and sensitization of previously uninvolved afferents (peripheral sensitization).

Another component of the syndrome is what is known as central sensitization. Central sensitization includes alteration of sensory processing in the brain, malfunction of descending pain inhibitory mechanisms, increased activity of pain facilitating pathways, and long-term potentiation of neuronal synapses in the anterior cingulate cortex. Immunological factors that induce peripheral nervous system dysfunction and activation of normally inactive fibers, including cytokines such as tumor necrosis factor α (TNF-α) and chemokines.

Several articles in the scientific literature describe the treatment of DPC-related syndromes such as vulvodynia, bladder pain syndrome, and myofascial pain.

In particular, the article by Murina F, Bianco V, Radici G, Felice R, Di Martino M, Nicolini U., “Transcutaneous electrical nerve stimulation to treat vestibulodynia: a randomized controlled trial”, BJOG. 2008; 115:1165-70 demonstrated that transcutaneous electrical nerve stimulation (TENS) is of significant benefit in the management of vulvar and sexual pain and vulvodynia.

Neuroinflammation plays a key role in vulvodynia and PCD syndrome. Current research focuses on strategies to modulate their activation based on natural mechanisms with the ability to self-defense against inflammation. Particularly promising molecules in clinical and animal studies include palmitoylethanolamide (PEA) and alphalipoic acid (ALA).

The study described in the article Murina F, Graziottin A, Felice R, Radici G, Tognocchi C, “Vestibulodynia: synergy between palmitoylethanolamide+transpolydatin and transcutaneous electrical nerve stimulation”, J Low Genit Tract Dis. 2013; 17:111-6 confirmed that TENS in combination with PEA is of significant benefit in the management of vestibulodynia, even in the home environment.

The study described in the article Murina F, Graziottin A, Felice R, Gambini D, “Alpha lipoic acid plus omega-3 fatty acids for vestibulodynia associated with painful bladder syndrome,” JOGC, 1 Mar. 2017, 39(3):131-137 evaluated the efficacy of alpha-lipoic acid (ALA) and omega-3 polyunsaturated fatty acids (omega-3 PUFAs) in combination with amitriptyline therapy in patients with vestibulodynia/painful bladder syndrome (VBD/PBS), demonstrating that the addition of omega-3 ALA/PUFA to amitriptyline treatment in patients with VBD+PBD appears to improve outcomes and may allow lower dosing of amitriptyline, resulting in fewer adverse effects.

Electroporation (EP) is the transient structural disruption of lipid bilayer membranes due to the application of high-voltage pulses. Its topical application has been shown to increase transdermal delivery of drugs of different orders of magnitude. In addition, EP expands the range of drugs (macromolecules, lipophilic or hydrophilic, charged or neutral molecules) that can be delivered transdermally. The use of electroporation is described i, for example, in US 2017/021025 A1.

The study published in the article Filippo Murina, Raffaele Felice, Stefania di Francesco and Silvia Oneda, “Transmucosal delivery of macromolecules using vaginal electroporation to treat vestibulodynia: A pilot study. Clin Obstet Gynecol Reprod Med, 2017, demonstrated the efficacy of EP applied via vaginal probe using a mix of two drugs (amitriptyline+lidocaine) in patients diagnosed with vestibulodynia.

Cannabis sativa L. (Cannabaceae) is a plant known since ancient times for its potential medicinal properties and its therapeutic uses.

Cannabis inflorescences or flos contain the highest concentration of cannabinoids, the active ingredients characteristic and exclusive to the Cannabis plant, the two main ones being delta-9-tetrahydrocannabinol (THC) and cannabidiol (CBD).

Currently, more than 500 substances have been recognized in the Cannabis plant, among which there are more than 120 cannabinoids, for example tetrahydrocannabinol (THC), cannabidiol (CBD), cannabichromene (CBC), cannabigerol (CBG), cannabidivarin (CBDV), cannabinol (CBN) and others, which are present in varying amounts in the aerial parts of the plant.

Cannabis and its derivatives are currently accredited for the treatment of a number of pathological conditions, including chemotherapy-induced pain, nausea and vomiting, spasticity associated with multiple sclerosis, and some forms of epilepsy (Abrams, D. I., European Journal of Internal Medicine, March 2018, Volume 49, Pages 7-11). Δ9-THC (trans-delta-9-tetrahydrocannabinol) is one of the main cannabinoids in Cannabis responsible for its therapeutic effects and uses, but its psychotropic activity has limited its therapeutic use. WO 2020/209902 A1 describes a suppository comprising cannabis oil for rectal or vaginal administration in the treatment of certain types of cancer. CN 108 853 079 A describes a pharmaceutical composition comprising cannabinoids for the treatment of irritable bowel syndrome.

Cannabinoids have been studied in various types of pain in vitro and in animal models. The cannabinoid receptors are CB1 and CB2. CB1 receptors are primarily found in the encephalon, particularly in the basal ganglia, globus pallidus, and substantia nigra, while CB2 receptors are expressed in cells of the immune system, peripheral nervous system, and glial cells of the central nervous system, with involvement in neuropathic pain. CB2 receptors are not normally present in dorsal root ganglia of nerves, but, following nerve injury, have been found in sensory neurons.

Cannabidiol (CBD) is the other main cannabinoid present in the cannabis plant which, unlike Δ9-THC, is devoid of psychotropic activity, (Babalonis et al., Drug Alcohol Depend. 2017 Mar. 1; 172: 9-13). The chemistry and pharmacology of CBD, as well as its molecular targets, have recently received significant attention in scientific research, which has highlighted its therapeutic potential, (Pisanti et al., Cannabidiol: State of the art and new challenges for therapeutic applications, Pharmacology and Therapeutics, Volume 175, July 2017, Pages 133-150) in areas such as immune-inflammatory, including disorders of the skin, mucous membranes and skin adnexa. Indeed, cannabidiol can have beneficial effects on the skin and interact with the skin's endocannabinoid system, regulating, for example, sebum secretion. Numerous scientific studies have confirmed the regenerative capacities of cannabidiol, which is also useful as an anti-inflammatory, antioxidant, cicatrizant and antibacterial agent for the skin and skin adnexa and to support natural cell renewal.

Cannabidiol (CBD) has also been reported to produce pain relief, although the cellular and molecular mechanisms underlying this effect remain unclear. It is known that the TRPV1 receptor integrates nociceptive stimuli and plays a key role in pain signaling, and in a recent in vitro study CBD was found to inhibit neuronal hypersensitivity following TRPV1 activation in sensory neurons in an animal model (Anand et al., “CBD Effects on TRPV1 Signaling Pathways in Cultured DRG Neurons,” Journal of Pain Research, 2020:13 2269-2278).

Cannabidiol is mainly extracted from the aerial parts of cannabis (Cannabis sativa L.), but is also produced synthetically.

It is well known that cannabidiol, both natural and synthetic, is poorly suited for the preparation of topical formulations, in particular cosmetic products and/or medical devices and/or pharmaceutical preparations for application to skin and mucous membranes, due to its poor solubility in water, and the consequent instability or incompatibility with the high percentage of water usually present in a cream, lotion, gel or other topical formulation. Cannabidiol is therefore currently used in oily solutions/suspensions, with a poor ability to penetrate through the skin and epithelia, which prevents or reduces the effectiveness of topical administration of cannabidiol.

US 2017/281481 A1 describes a topical composition comprising plant extracts incorporated into niosomes. Patent application No. 1020200004450 describes a composition comprising cannabidiol incorporated into niosomes, preferably having a size of less than 500 nm for use in cosmetic products and/or medical devices and/or pharmaceutical preparations for skin and mucosal applications.

### SUMMARY OF THE INVENTION

The Applicant addressed the issue of treatment of syndromes associated with pelvic pain.

After extensive experimentation, Applicant found that the use of the composition described in patent application Ser. No. 10/2020000004450, particularly in gel form, carried significant benefits, particularly when combined for application to a dynamic electroporation apparatus, in the treatment of vestibulodynia and chronic pelvic pain.

Specifically, the Applicant found that administration of a gel formulation comprising cannabinoids brought significant improvement in vulvar and/or pelvic pain symptoms—burning and/or pain—in patients with vestibulodynia and chronic pelvic pain.

The Applicant further found that such improvement occurred in 60% of the patients treated, even after only two treatment sessions, and preferably after between three and six sessions.

The Applicant ultimately found that none of the treated patients exhibited any adverse side effects, demonstrating that the treatment was well tolerated.

Thus, a first object of the present invention relates to a topical pharmaceutical formulation comprising an aqueous-based composition comprising cannabinoids, in particular cannabidiol, in niosomes having a size of less than 500 nm, and at least one topically acceptable excipient, for use in the treatment of syndromes associated with chronic pelvic pain, in particular by topical application by means of a dynamic electroporation apparatus.

A second aspect of the present invention relates to a method for treating syndromes associated with chronic pelvic pain by topical application of a pharmaceutical formulation comprising cannabinoids in accordance with the first aspect of the present invention by means of a dynamic electroporation apparatus.

### BRIEF DESCRIPTION OF THE FIGURES

The present invention will be best illustrated in the following detailed description, set forth below with reference to the accompanying drawings, provided for illustrative purposes only and, therefore, not limiting, in which:

FIG. 1 shows a longitudinal cross-sectional view of the vaginal applicator of a dynamic electroporation device, and

FIG. 2 is a schematic representation of the dynamic electroporation device as a whole.

### DETAILED DESCRIPTION OF THE INVENTION

The present invention relates in a first aspect to a topical pharmaceutical formulation comprising an aqueous-based composition comprising cannabinoids, in particular cannabidiol, in niosomes having a size of less than 500 nm, and at least one topically acceptable excipient, for use in the treatment of syndromes associated with chronic pelvic pain.

The syndromes associated with chronic pelvic pain are a set of symptoms mainly characterized by chronic pain in the pelvis and/or perineum, with possible irradiation to the lumbar region, to the external genitalia, vagina and vulva, to the groin, to the suprapubic region, to the sacro-coccyx, to the root of the thighs

Syndromes associated with chronic pelvic pain that may find relief upon treatment with the topical pharmaceutical formulation of the present invention are vulvodynia, vestibulodynia, chronic prostatitis and prostatodynia, abacterial cystitis, painful bladder syndrome, urethral syndrome, dyspareunia, anus elevator syndrome, myofascial pain, coccicodynia or coccygodynia, proctalgia fugax and pudendal neuralgia.

Preferably, said niosomes comprise (i) at least one linear or branched polyglycerol esterified with saturated or monounsaturated linear fatty acids, (ii) at least one polysaccharide, and optionally (iii) at least one glycol having 4 to 16 carbon atoms.

The term “topically acceptable” as used herein is intended to define substances recognized as having no adverse side effects (irritation, toxicity, and so forth) when applied to the skin, epithelia, and/or mucous membranes.

The expression “niosome” or “niosomes” as used in the present description is intended to define hydrophilic vesicles formed by one or more non-ionic surfactants oriented in a bilayer.

Advantageously, the niosomes used in the present invention have diameters less than 400 nm, more preferably less than 300 nm, and even more preferably less than 250 nm.

Preferably, the niosomes used in the present invention have diameters greater than 50 nm, more preferably greater than 70 nm, and even more preferably greater than 90 nm.

Advantageously, the niosomes used in the present invention have diameters between 110 nm and 220 nm, preferably between 150 nm and 200 nm.

The linear or branched polyglycerol esterified with saturated or monounsaturated linear fatty acids useful in the present invention is obtained by esterification of a linear or branched polyglycerol with saturated or monounsaturated linear fatty acids.

The cannabidiol useful in the present invention may be of natural and/or synthetic origin, preferably having a purity of 95% or greater, 96% or greater, 97% or greater, 98% or greater, or 99% or greater. Advantageously, the cannabidiol useful in the present invention has a purity of about 100%.

Naturally occurring cannabidiol may contain minority percentages of other cannabinoids, such as, for example, cannabichromene, cannabigerol, and cannabinol, generally in the aggregate equal to or less than 5%, equal to or less than 4%, equal to or less than 3%, equal to or less than 2%, or equal to or less than 1%.

Preferred examples of polyglycerols include triglycerol, tetraglycerol, hexaglycerol, octaglycerol, decaglycerol. Linear or branched polyglycerols useful in the present invention are commercially available.

Examples of commercial products are polyglycerols distributed by American International Chemical LLC under the trade name Polyglycerol-3, by Spiga Nord S.p.A. under the trade names Vegetable Polyglycerine-3, Vegetable Polyglycerine-4, Vegetable Polyglycerine-6, and Vegetable Polyglycerine-10, and by Solvay Chemicals, Inc. under the trade names Polyglycerol-3 and Polyglycerol-4.

Useful examples of saturated linear fatty acids include monocarboxylic acids having 4 to 32 carbon atoms, such as butyric acid, valeric acid (valeric), capronic acid, enanthic acid, caprylic acid, pelargonic acid, capric acid, lauric acid, myristic acid, palmitic acid, margaric acid, stearic acid, arachic acid, beenic acid, lignoceric acid, cerotic acid, montanic acid, melissic acid, and laceroic acid.

Preferred linear saturated fatty acids include monocarboxylic acids having 12 to 22 carbon atoms, such as lauric acid, myristic acid, palmitic acid, margaric acid, stearic acid, arachic acid, and beenic acid.

Useful examples of linear monounsaturated fatty acids include monocarboxylic acids having 14 to 24 carbon atoms, such as for example myristoleic acid, palmitoleic acid, oleic acid, gadoleic acid and erucic acid.

Useful examples of mixtures of fatty acids include vegetable oils obtainable by pressing or extracting seeds or fruits, for example, olive oil, peanut oil, coconut oil, palm oil, and canola oil. Due to the reduced content of polyunsaturated acids, olive and coconut oils, and in particular olive oil, are preferred.

Esters of linear or branched polyglycerols with linear saturated or monounsaturated fatty acids, or mixtures thereof, useful in the present invention are commercially available. Examples of commercial products include polyglycerol esters made and distributed by the company Lonza under the trade name Polyaldo, such as for example Polyaldo® 6-2-S [Polyglyceryl-6 Disterate], Polyaldo®10-1-S [Polyglyceryl-10 Stearate], Polyaldo® 10-1-O [Polyglyceryl-10 Oleate], Polyaldo® 10-2-P [Polyglyceryl-10 Dipalmitate], by the company Hydrior AG, Germany under the trade name Hydriol, as for example HYDRIOL ° PGO (Polyglycerol-4-oleate), HYDRIOL ° PGD (Polyglycerol-3-diisostearate) by the company Naturalis s.r.l. under the trade name Soavirol, such as Soavirol OV6 (olive oil polyglyceryl-6 ester), Soavirol OV4 (olive oil polyglyceryl-4 ester), and by the company Nikko Chemicals Co, Ltd. under the trade name Nikkol Hexaglyn, such as for example Nikkol Hexaglyn 1-L (polyglyceryl-6 laurate) and Nikkol Hexaglyn PR-15 (polyglyceryl-6 polyricinoleate).

The polysaccharide useful in the present invention is preferably chosen from the group consisting of polysaccharides of natural origin, such as for example pullulans, glucans, alginates, amylose, glycogen, inulin, and so on.

Advantageously, the polysaccharide useful in the present invention is chosen from the group consisting of alpha- and beta-glucans, more preferably beta-glucans.

Beta-glucans are linear polysaccharides consisting of glucose molecules joined together by β(1-3) glycosidic bonds. Beta-glucans are natural products found in cereals, bacteria and fungi. Oats and barley are particularly rich in beta-glucans, and their production comes mainly from extraction from these cereals. Beta-glucans obtained from fungi are also widely available commercially. Beta-glucans obtained from fungi and yeast contain joined branches with glycosidic and β(1-6) bonds, whereas beta-glucans obtained from cereals have glycosidic and β(1-3) and β(1-4) bonds. Cereal-derived beta-glucans are more soluble in water, and are therefore preferred for the purposes of the present invention.

Beta-glucans particularly useful for the purposes of the present invention are beta-glucans distributed by the company Ohly GmbH, Germany, under the trade name Ohly-GO ° Glucan, by the company Maypro Industries LLC under the trade name Chitoglucan®, by the company Lesaffre Human Care under the trade name Lynside® Wall Glucan, and by the company HerbaKraft Inc. under the trade name Beta Glucan.

The glycol having 4 to 16 carbon atoms useful in the present invention is preferably selected from the group consisting of 1,2-butanediol, 1,2-pentanediol, 1,2-hexanediol, 1,2-heptanediol, 1,2-octanediol (caprilyl glycol), 1,2-decanediol (capril glycol), 1,2-dodecanediol (lauryl glycol), and 1,2-hexadecanediol.

1,2-hexanediol, 1,2-heptanediol, 1,2-octanediol (caprilyl glycol), 1,2-decanediol (capril glycol) are particularly preferred. For the purposes of the present invention, 1,2-octanediol (caprylyl glycol) is advantageously used.

Commercial products comprising glycols useful for the purposes of the present invention are distributed by the company CBW Chemie GmbH, Germany, by the company Inolex Inc., USA under the trade name Lexgard °, by the company Wintersun Chemical, USA, and by the company Chemos GmbH & Co. KG.

In the realization of the niosomes used for the purposes of the present invention, further components suitable for stabilizing and preserving the aqueous solution/dispersion of niosomes are preferably used, such as for example water-soluble natural antioxidants such as ascorbic acid and its derivatives, and polyphenols.

The aqueous composition comprising niosomes can be prepared according to techniques known in the art, for example, by hand shaking or ultrasonic technique.

The manual agitation technique comprises a first phase of solubilization of the components in a volatile organic solvent, such as, for example, ethyl ether, chloroform or methanol, conducted in a glass flask, a second phase of evaporation, conducted in a rotary evaporator at room temperature (20°−25° C.) which leaves a thin layer of the components deposited on the walls of the flask, and finally a third phase of rehydration with an aqueous phase comprising the plant extracts at a temperature between 0° and 60° C. under slight agitation.

The ultrasonic technique comprises sonication at a temperature between 0° and 60° C. of a dispersion obtained by mixing an organic phase comprising surfactants and an aqueous phase comprising plant extracts.

These and other methods of preparing compositions comprising niosomes are described in the literature, for example in the article by Madhav et al, “Niosomes: a novel drug delivery system”; International journal of research in pharmacy and chemistry, IJRPC 2011, 1(3), 498-5.11.

The dispersion/iosome solution of the present invention preferably comprises the amounts of components described below and expressed as a weight percentage of the total weight of the dispersion/iosome solution (% w/w).

The cannabidiol is included in the dispersion/niosome solution up to an amount of 10% w/w. Preferably, the cannabidiol is present in an amount between 1% and 8% w/w, more preferably between 2% and 7% w/w, and even more preferably between 3% and 6% w/w. Advantageously, the amount of cannabidiol is between 4% and 5% w/w.

The dispersion/niosome solution comprises an amount of polyglycerol esters between 40% and 90% w/w, preferably between 50% and 80% w/w.

The amount of beta-glucan included in the dispersion/niosome solution is between 1% and 5% w/w, preferably between 1% and 3% w/w.

The amount of glycol included in the dispersion/niosome solution is between 0.1% and 5% w/w, preferably between 0.5% and 3% w/w.

The resulting niosome dispersion/solution comprises water in an amount of between 10% and 40% w/w, preferably between 20% and 30% w/w.

The niosome dispersion/solution may comprise other components, such as stabilizers and preservatives, up to the amount of 1% w/w.

The topical pharmaceutical formulation of the present invention may be liquid or semi-solid.

In particular, the topical pharmaceutical formulation of the present invention comprises a pharmaceutical preparation and/or a medical device, for application to the skin, epithelia and mucous membranes.

The topical pharmaceutical formulation of the present invention advantageously comprises liquid or semi-solid formulations within which the aqueous-based niosome composition is dispersed in an amount of between 0.5% and 20% by weight, preferably between 1% and 15% by weight, and more preferably between 2% and 10% by weight, relative to the total weight of the topical formulation.

Liquid formulations of the present invention include solutions, emulsions, microemulsions, lotions, gels, foams, milks, micellar waters, oils, tensiolites or suspensions having a wide variation in viscosity.

Liquid formulations include, for example, aqueous solutions, hydro-alcoholic solutions, oily solutions, emulsions obtained by dispersion of an oily phase in an aqueous phase (oil-in-water) or, vice versa, of an aqueous phase in an oily phase (water-in-oil), and suspensions, obtained by dispersion of a dispersed phase, consisting of solid particles, in a dispersing medium generally represented by an aqueous or oily liquid of a certain viscosity.

Semi-solid formulations of the present invention include creams, gels, balms, ointments, pastes, creme-gel, sticks and waxes.

Advantageously, the topical pharmaceutical formulation of the present invention is a gel or a cream-gel.

In addition, the topical pharmaceutical formulation of the present invention may comprise various topically acceptable additives or vehicles useful in the preparation of pharmaceutical preparations and/or medical devices known to the technician in the art, such as, for example, emulsifiers, moisturizers, solvents, emollients, stabilizers, viscosifiers, preservatives, lubricants, sequestering or chelating agents, fillers, powders, fragrances, perfumes, absorbents, colorants and opacifiers, antioxidants, vitamins, natural extracts, polysaccharides, shielding substances, UV filters, essential oils, keratin-active substances, and amino acids.

Suitable additive solvents include, for example, water, alcohols, ketones (such as acetone and methyl isobutyl ketone), glycols (such as ethylene glycol, propylene glycol and butylene glycol), polyethylene glycols (such as PEG-40, PEG-50, PEG-60), alkyl acetates (such as amyl acetate, isopropyl acetate, butyl acetate), paraffins and isoparaffins, cycloalkyls (such as cyclohexane), glycerol, natural and synthetic oils, natural and synthetic triglycerides, essential oils.

Advantageously, the topical pharmaceutical formulation of the present invention is an aqueous formulation.

In an aqueous formulation, the water represents the main component of a formulation even reaching an amount of up to 99% by weight relative to the weight of the total formulation. The aqueous formulation preferably contains an amount of water between 25% and 99%, preferably between 35% and 95%, and more preferably between 50% and 90% by weight relative to the weight of the total aqueous formulation.

The aqueous formulation of the present invention may preferably comprise a total amount of non-aqueous solvents between about 0.1% and about 60%, more preferably between 1% and 40%, and even more preferably between 5% and 35% by weight relative to the weight of the total formulation.

Examples of suitable emulsifying additives include non-ionic, cationic, anionic and amphoteric surfactants, or a combination thereof. Examples of emulsifiers useful in the present invention are sorbitans, ethoxylated long-chain alcohols, alkyl polyglycosides, soaps, alkyl sulfates, such as, for example, sodium cetylstearyl sulfate, monoalkyl and dialkyl phosphates, alkyl sulfonates, hydrogenated castor oil, acyl isothionates, sucrose esters, betaines, lecithins, quaternary ammonium salts, alkyloleates, glycerides, such as, for example, caprylocaproyl polyoxylglycerides (caprylocaproyl macrogolglycerides) and emulsifiers from olive oil.

Preferably, the topical pharmaceutical formulation of the present invention comprises a total amount of emulsifiers between about 0.1% and about 60%, more preferably between 0.5% and 25%, and even more preferably between 0.5% and 10% by weight relative to the weight of the total formulation.

Typical viscosity additives useful in the present invention are, for example, xanthan gum, hydroxypropylcellulose, hydroxyethylcellulose, carbopol, carrageenans, polyoxamers, and acacia gum.

Advantageously, the topical pharmaceutical formulation of the present invention comprises a total amount of viscosifiers between about 0.1% and about 25%, more preferably between 0.5% and 10%, and even more preferably between 0.5% and 5% by weight relative to the weight of the total formulation.

Examples of additives with moisturizing action useful in the present invention are, for example, urea, allantoin, hyaluronic acid and its derivatives, glycerin, amino acids, acetylmonoethanolamide, butoxypropanol, butyl glycol, low molecular weight polyethylene glycols (such as PEG-40, PEG-50, PEG-60), aloe, mallow, trehalose and sorbitol.

Preferably, the topical pharmaceutical formulation of the present invention comprises a total amount of moisturizers between about 0.05% and about 25%, more preferably between 0.5% and 10%, and even more preferably between 0.1% and 5% by weight relative to the weight of the total formulation.

Examples of suitable emollient additives useful in the present invention include, for example, lanolin, almond oil, olive oil, vegetable oils, jojoba oil, argan oil, hydrogenated castor oil, natural lipophilic extracts, microcrystalline wax, polydimethylsiloxane (dimethicone), polymethylphenylsiloxane, glycol and silicone polymers, mineral oils, paraffin, ozocherite, ceresin, triglyceride esters, acetylated monoglycerides, ethoxylated glycerides, alkyl esters of fatty acids, fatty acids, long chain alcohols, sterols, beeswax, polyhydric alcohols, polyesters, and fatty acid amides.

Preferably, the topical pharmaceutical formulation of the present invention comprises a total amount of emollients between about 0.1% and about 25%, more preferably between 0.5% and 10%, and even more preferably between 0.5% and 5% by weight relative to the weight of the total formulation.

Examples of fragrances useful in the present invention are, for example, natural essential oils or fractions or concentrates of essential oils, such as, for example, lemon oil, bergamot oil, lavender oil, limonene, linalool. Preferably, the topical pharmaceutical formulation of the present invention comprises a total amount of fragrances between about 0.001% and about 0.1%.

Examples of suitable preservative additives useful in the topical pharmaceutical formulation of the present invention include, for example, alcohols, such as ethanol, phenoxyethanol and benzyl alcohol, methyl and propyl parahydroxybenzoate, butylated hydroxyanisole (BHA), sorbates, urea derivatives, and isothiazolinones, natural preservatives, such as ascorbic acid and derivatives, tocopherol and derivatives, polyphenols.

Preferably, the topical pharmaceutical formulation of the present invention comprises a total amount of preservatives between about 0.01% and about 2.00%, more preferably between 0.05% and 1.00%, and even more preferably between 0.1% and 0.5% by weight relative to the weight of the total formulation.

Examples of sequestrant or chelating additives useful in the present invention are EDTA, HEDTA, alkyl oxalates, lithium or potassium oxalate, sodium or potassium pyrophosphate. Preferably, the topical pharmaceutical formulation of the present invention comprises a total amount of sequestering or chelating additives between about 0.01% and about 20%, more preferably between 0.05% and 10%, and even more preferably between 0.1% and 5% by weight relative to the weight of the total formulation.

Examples of suitable stabilizing additives useful in the present invention are long chain alcohols (such as cetyl alcohol, stearyl alcohol) and mixtures thereof, high molecular weight polyethylene glycols (such as PEG-9000 and PEG 14000) and polyvinylpyrrolidones (such as povidone).

The topical pharmaceutical formulation of the present invention preferably comprises a total amount of stabilizers between about 0.1% and about 25%, more preferably between 0.5% and 15%, and even more preferably between 1% and 10% by weight relative to the weight of the total formulation.

Examples of suitable powder additives useful in the present invention are elastomeric silicones such as dimethicone/vinyl dimethicone crosspolymers (DC 9506, Dow-Corning), mixtures of cyclomethicone and dimethicone crosspolymers (DC 9040, Dow Corning), silica treated crosspolymers of dimethicone and vinyl dimethicone (DC 9701, Dow Corning), mixtures of crosspolymers of cyclomethicone and dimethicone/vinyldimethicone (SFE 839, GE Bayer Silicones).

The topical pharmaceutical formulation of the present invention preferably comprises a total amount of powder additives between about 0.1% and about 5%, more preferably between 0.2% and 1%, by weight relative to the weight of the total formulation.

Examples of opacifying agents useful in the present invention are zinc or aluminum oxide, titanium or zinc dioxide, alumina, mica, fatty acid salts with aluminum, and gypsum.

Examples of dyes preferably employed in the present invention are easily washable water-soluble dyes that do not stain the skin and leave no residue such as, for example, Acid Blue 3 C.I.42051, Acid Blue 9 C.I.42090, Acid Blue 74 C.I.73015, Pigment Blue 15 C.I.74160, Acid Yellow 3 C.I.47005, Food Yellow 3 C.I.15985, Acid Yellow 23 C.I.19140, Acid Yellow 73 C.I.45350, Acid Red 14 C.I.14720, Acid Red 18 C.I.16255, Acid Red 27 C.I.16185, Acid Red 51 C.I.45430, Acid Green 1 C.I.10020, Acid Green 25 C.I.61570, and mixtures thereof.

Preferably, the topical pharmaceutical formulation of the present invention comprises a total amount of opacifying and coloring agents between about 0.01% and about 15%, more preferably between 0.05% and 5% by weight relative to the weight of the total formulation.

Preferably, the topical pharmaceutical formulation of the present invention may comprise UV filters capable of shielding the skin from the action of ultraviolet radiation. Examples of UV filters are, for example, acrylates such as 2-ethylhexyl 2-cyano-3,3-diphenylacrylate (PARSOL 340) and ethyl 2-cyano-3,3-diphenylacrylate, camphor derivatives such as camphor 4-methyl benzylidene (PARSOL 5000), and camphor 3-benzylidene, cinnamates such as octyl methoxycinnamate (PARSOL MCX), ethoxyethyl methoxycinnamate, diethanolamine methoxycinnamate (PARSOL Hydro), triazone derivatives such as ethylhexyl triazone (UVINUL T-150), diethylhexyl butamido triazone (UVASORB HEB), dibenzoylmethane derivatives such as 4-tert-butyl-4′-methoxydibenzoylmethane (PARSOL 1789), dimethoxydibenzoylmethane, benzotriazole derivatives such as 2,2′-methylene-bis-(6-(2H-benzotriazol-2-i1)-4-(1,1,3,-tetramethylbutyl)-phenol (TINOSORB M), triazine derivatives as bis-ethylhexyloxyphenol methoxyphenyl triazine (TINOSORB S).

Preferably, the topical pharmaceutical formulation of the present invention comprises a total amount of UV filters between about 0.1% and about 20%, more preferably between 0.5% and 15% by weight relative to the weight of the total formulation.

A second aspect of the present invention relates to a method for treating syndromes associated with chronic pelvic pain by topical application of a pharmaceutical formulation comprising cannabinoids in accordance with the first aspect of the present invention by means of a dynamic electroporation apparatus.

Electroporation is a non-invasive and painless practice that uses light electrical impulses to allow the active ingredient used to penetrate deeper into the skin and/or mucosa treated.

The active ingredients are conveyed with a device consisting of a handpiece equipped with electrodes, which, through a low-voltage electrical discharge, favours the dilation of the skin pores and the creation of small channels. Through the latter, the active ingredients reach the deeper, underlying layers.

Electroporation apparatuses are known commercially and essentially comprise an applicator or handpiece comprising a reservoir to contain the active ingredient to be conveyed and a connection to an electrical pulse generator and a control system with a viewer.

Electroporation devices and methods particularly useful for applying the topical pharmaceutical composition of the present invention are described, for example, in WO2013117306 and WO2009130258A1. Other electroporation devices and methods are described in WO0181532A1, WO03104448A1, WO2009016662A1, WO2012147072A1 and WO2014066655A2.

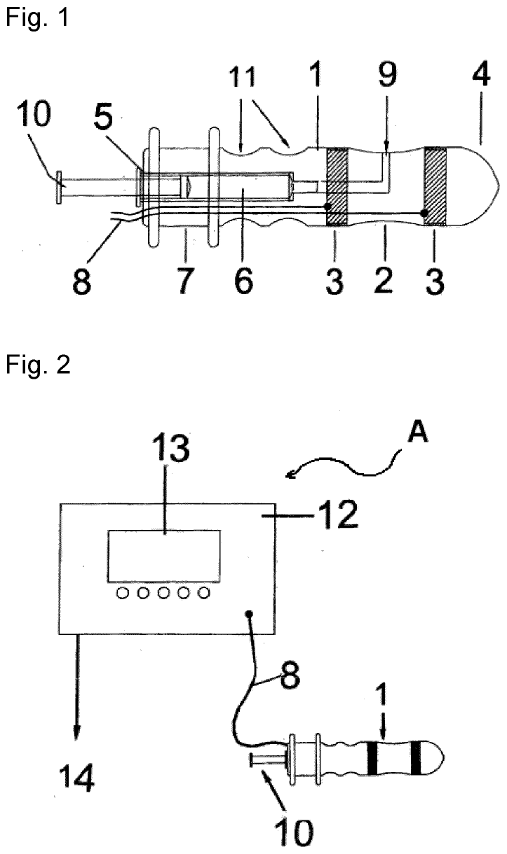

An example electroporation apparatus is schematically illustrated in FIGS. 1 and 2, wherein FIG. 1 represents a longitudinal cross-sectional view of the vaginal applicator of the dynamic electroporation apparatus, and FIG. 2 schematically represents the apparatus as a whole.

With reference to the figures, the apparatus (A) substantially comprises an applicator (1) made of insulating material, rounded in the front part (4) to facilitate an easy and painless introduction and adherence with the internal vaginal wall to be treated. In the intermediate front part, in addition to the introductory part, the applicator comprises a partially convex area forming a substantially annular recess (2) at the sides of which are arranged two rings (3), made of surgical steel or conductive plastic material, electrically connected to the electric pulse generator (12)

At the rear there is a central seat (5) comprising an anterior coaxial conduit with at least one radial outlet (9) flowing into the substantially central area of said convex annular recess (2). In the seat (5) there is positionable a needle-free syringe (6), with a plunger (10) thereof, intended to contain the topical pharmaceutical formulation of the present invention.

Acting on the plunger (10), the fluid product is pushed along the duct (9) and discharged into the convex annular recess (2) where it can expand and uniformly come into contact with the whole vaginal surface to be treated, included between the electrodes (3) which, powered by the electric impulse generator (12), perform the electroporation action necessary to favour the uniform and deep transdermal and transmucosal vehiculation of the topical pharmaceutical formulation of the present invention.

A handle (7) is provided at the rear intended to facilitate the introduction, positioning, release action of the topical pharmaceutical formulation of the present invention and removal of the ‘applicator.

Two circumferential convexities (11) in the central part of the applicator (1) are used to calibrate the introduction depth and make it easier to hold the applicator in position without additional manual support.

In the rear part (7) of the applicator there is also provided the output of the connector or of the cables (8) for connecting the two electroporation electrodes (3) to a programmable electric impulse generator (12), command and control with display (13), which can be powered by batteries or by a fixed network (14).

An example of a commercial electroporation device useful for the purposes of the present invention is the EPV® device manufactured by the company Blue-Moon Srl (https://www.blue-moon.it/) and distributed by the company Santec Srl (https://www.santeclaser.it).

The following experimental portion illustrates at least one way of performing the invention, but does not in any way restrict the scope of protection as defined in the claims appended hereto.

### EXPERIMENTAL PART

### Example 1—Preparation of Niosomes

A dispersion of niosomes (CBD-S5) comprising cannabidiol (CBD) was prepared with the composition shown in Table 1 below. The resulting composition had the appearance of a uniform pale yellow viscous gel. The weight percentage relative to the weight of the total composition is expressed for each component.

TABLE 1

| Component | CBD-S5 |
|---|---|
| Cannabidiol | 5 |
| Polyglyceryl-6 oleate | 30 |
| Polyglyceryl-10 caprate | 20 |
| Polyglyceryl-4 caprate | 20 |
| Beta-glucan | 2 |
| Caprylyl glycol | 1 |
| Water | to 100 g |

### Example 2—Preparation of the Topical Pharmaceutical Formulation in 5% Gel

The niosome dispersion (CBD-S5) prepared in Example 1 was used for the preparation of a topical pharmaceutical formulation in the form of a gel by electroporation according to the following procedure.

A dispersion of carbomer (5 g, Carbopol ° Ultrez 10, from Lubrizol) in demineralised water is added to a dispersion of high viscosity hydroxyethylcellulose (2 g, Natrosol™ 250 HHR, from Ashland) in propanediol (20 g, Zemea®, from DuPont Tate & Lyle Bio Products) and water, stirring vigorously. A preservative mixture (17 g/, Sensiva PA30®, from Schülke & Mayr GmbH) dispersed in water (30 g) is added to the resulting dispersion and then neutralized with a 30% sodium hydroxide solution to pH 6.8. To the resulting gel, the niosome dispersion (50 g) is slowly added under stirring, followed by a 20% sodium chloride solution (6 g).

The resulting topical gel pharmaceutical formulation has the following composition:

| Component | CBD Gel |
|---|---|
| Propanediol | 20 |
| Sensiva PA30 ® (*) | 17 |
| Carbomer | 5 |
| Hydroxyethylcellulose | 2 |
| Polyglyceryl-6 oleate | 15 |
| Polyglyceryl-10 caprate | 10 |
| Polyglyceryl-4 caprate | 10 |
| Sodium chloride | 1.0 |
| Cannabidiol | 2.5 |
| Beta-glucan | 1.0 |
| Caprylyl glycol | 0.5 |
| Sodium hydroxide | to pH 6.8 |
| Water | to 1000 g |

(*) Mixture of propanediol (8 g), 2-phenylethyl alcohol (6 g), undecyl alcohol (2.5 g) and tocopherol (0.5 g)

### Example 3—Preparation of Topical Gel Pharmaceutical Formulation 6%

The niosome dispersion (CBD-S5) prepared in Example 1 was used for the preparation of a topical pharmaceutical formulation in the form of a gel by electroporation according to the following procedure.

A dispersion of carbomer (5 g, Carbopol ° Ultrez 10, from Lubrizol) in demineralised water is added to a dispersion of high viscosity hydroxyethylcellulose (2 g, Natrosol™ 250 HHR, from Ashland) in propanediol (20 g, Zemea®, from DuPont Tate & Lyle Bio Products) and water, stirring vigorously. A preservative mixture (17 g, Sensiva PA30®, from Schülke & Mayr GmbH) dispersed in water (30 g) is added to the resulting dispersion and then neutralized with a 30% sodium hydroxide solution to pH 6.8. To the resulting gel, the niosome dispersion (60 g) is slowly added under stirring, and then a 20% sodium chloride solution (6 g) is added.

The resulting topical gel pharmaceutical formulation has the following composition:

| Component | CBD-S5 |
|---|---|
| Propanediol | 20 |
| Sensiva PA30 ® (*) | 17 |
| Carbomer | 5 |
| Hydroxyethylcellulose | 2 |
| Polyglyceryl-6 oleate | 15 |
| Polyglyceryl-10 caprate | 10 |
| Polyglyceryl-4 caprate | 10 |
| Sodium chloride | 1.0 |
| Cannabidiol | 2.5 |
| Beta-glucan | 1.0 |
| Caprylyl glycol | 0.5 |
| Sodium hydroxide | to pH 6.8 |
| Water | to 1000 g |

(*) Mixture of propanediol (8 g), 2-phenylethyl alcohol (6 g), undecyl alcohol (2.5 g) and tocopherol (0.5 g)

### Example 4—Clinical Protocol and Results

The formulation in Example 2 was used to treat 22 patients with vestibulodynia or chronic pelvic pain syndrome.

Patients were treated by application of the formulation of Example 2 delivered via vestibular or vaginal electroporation.

For each treatment session, 3 ml of the formulation from Example 2 was used.

Variation in vulvar and/or pelvic pain symptoms—burning and/or pain—were assessed with a visual analogue scale (VAS) scored from 0 to 10, where 0 meant “no pain” and 10 meant “maximum pain”.

The resulting score, in terms of percentage change, was evaluated according to the following criteria:
unchanged: no change or reduction equal to or less than 30%,
improved: reduction greater than 30% but equal to or less than 60%,
much improved: 60% greater reduction.

The results are summarized in Table 1 below.

TABLE 1

| | | Type | Number of | |
|---|---|---|---|---|
| Patient | Pathology | of EP | sessions | Outcome |
| 1 | DPC | EPvag | 5 | Improved |
| 2 | VBD | EPvest | 3 | Improved |
| 3 | DPC | EPvag | 4 | Much improved |
| 4 | DPC | EPvag | 4 | Improved |
| 5 | DPC | EPvag | 3 | Unchanged |
| 6 | VBD | EPvest | 4 | Improved |
| 7 | DPC | EPvag | 6 | Much improved |
| 8 | VBD | EPvest | 2 | Unchanged |
| 9 | DPC | EPvag | 3 | Unchanged |
| 10 | VBD | EPvest | 4 | Much improved |
| 11 | VBD | EPvest | 6 | Much improved |
| 12 | VBD | EPvest | 3 | Improved |
| 13 | DPC | EPvag | 6 | Improved |
| 14 | DPC | EPvag | 3 | Unchanged |
| 15 | DPC | EPvag | 2 | Unchanged |
| 16 | DPC | EPvag | 2 | Unchanged |
| 17 | DPC | EPvag | 2 | Improved |
| 18 | DPC | EPvag | 4 | Unchanged |
| 19 | VBD | EPvest | 2 | Unchanged |
| 20 | DPC | EPvag | 3 | Unchanged |
| 21 | VBD | EPvest | 3 | Improved |
| 22 | VBD | EPvest | 3 | Improved |

EPvag = vaginal electroporation
EPvest = vestibular electroporation
VBD = vestibulodynia
DPC = chronic pelvic pain

The results in Table 1 show that a total of 13 patients (including 7 with vestibulodynia and 6 with chronic pelvic pain) reported improvement or strong improvement, with a reduction in vulvar and/or pelvic pain symptoms—burning and/or pain—between 30% and 60% or greater than 60%, while 9 patients reported a reduction of less than 30% or unchanged symptoms.

Surprisingly, these results were manifested in some cases with only two or three sessions, although optimal results were obtained with between 4 and 6 sessions.

None of the treated patients reported any side effects from the administration of the Example 2 formulation by electroporation, confirming that the treatment was advantageously well tolerated.

### Example 5—Clinical Protocol and Results

The formulation of Example 3 was used to treat an additional 13 patients with vestibulodynia or chronic pelvic pain syndrome, in a double-blind cross-over study versus placebo, represented by the gel without the cannabidiol.

Patients were treated by application of the formulation of Example 3 delivered via vestibular or vaginal electroporation, and subjected to a series of consecutive treatments, from a minimum of 2 to a maximum of 6.

For each treatment session, 3 ml of the formulation of Example 3 was used.

Variation in vulvar and/or pelvic pain symptoms—burning and/or pain—were assessed with a visual analogue scale (VAS) scored from 0 to 10, where 0 meant “no pain” and 10 meant “maximum pain”.

The resulting score, in terms of percentage change, was evaluated according to the following criteria:
unchanged: no change or reduction equal to or less than 30%,
improved: reduction greater than 30% but equal to or less than 60%,
much improved: 60% greater reduction.

The results are summarized in Table 2 below.

TABLE 2

| | | Type | Number of | |
|---|---|---|---|---|
| Patient | Pathology | of EP | sessions | Outcome |
| 23 | DPC | EPvag | 4 | Much improved |
| 24 | DPC | EPvag | 4 | Improved |
| 25 | VBD | EPvag | 3 | Improved |
| 26 | VBD | EPvest | 3 | Improved |
| 27 | DPC | EPvag | 3 | Unchanged |
| 28 | DPC | EPvag | 6 | Improved |
| 29 | VBD | EPvest | 4 | Much improved |
| 30 | DPC | EPvag | 2 | Improved |
| 31 | DPC | EPvag | 4 | Improved |
| 32 | VBD | EPvest | 2 | Unchanged |
| 33 | DPC | EPvag | 3 | Improved |
| 34 | VBD | EPvest | 3 | Improved |
| 35 | DPC | EPvag | 6 | Much improved |

EPvag = vaginal electroporation
EPvest = vestibular electroporation
VBD = vestibulodynia
DPC = chronic pelvic pain

The results in Table 2 show that a total of 11 patients (including 4 with vestibulodynia and 7 with chronic pelvic pain) reported improvement or strong improvement, with a reduction in vulvar and/or pelvic pain symptoms—burning and/or pain—between 30% and 60% or greater than 60%, while only one patient reported a reduction of less than 30% or unchanged symptoms.

Surprisingly, these results were manifested in some cases with only two or three sessions, although optimal results were obtained with between 4 and 6 sessions.

None of the treated patients reported any side effects from the administration of the Example 3 formulation by electroporation, confirming that the treatment was advantageously well tolerated.

The Applicant surprisingly observed that the use of formulation 3, comprising a niosome concentration of 6% by weight, provided positive results on a greater number of treated patients than the use of formulation 2, comprising a niosome concentration of 5% by weight. This observation allowed to hypothesize the possible use of formulations with even higher concentrations of niosomes, between 8 and 10% by weight.

1. A topical pharmaceutical formulation, comprising:
a water-based composition comprising cannabinoids in niosomes, and
at least one topically acceptable excipient,
wherein the niosomes have a size of less than 500 nm.

2. The topical pharmaceutical formulation according to claim 1, wherein the niosomes comprise:
(i) at least one linear or branched polyglycerol, esterified with saturated or monounsaturated linear fatty acids, and
(ii) at least one polysaccharide.

3. The topical pharmaceutical formulation according to claim 1, wherein the cannabinoids are of natural or synthetic origin.

4. The topical pharmaceutical formulation according to claim 2, wherein the at least one linear or branched polyglycerol is at least one selected from the group consisting of triglycerol, tetraglycerol, hexaglycerol, octaglycerol, and decaglycerol.

5. The topical pharmaceutical formulation according to claim 2, wherein the saturated or monounsaturated linear fatty acids are selected from the group consisting of monocarboxylic acids having from 4 to 32 carbon atoms.

6. The topical pharmaceutical formulation according to claim 2, wherein the at least one polysaccharide is of natural origin.

7. The topical pharmaceutical formulation according to claim 1, wherein the niosomes comprise (iii) at least one glycol having 4 to 16 carbon atoms.

8. The topical pharmaceutical formulation according to claim 1, wherein the formulation is in a liquid or semi-solid form.

9. The topical pharmaceutical formulation according to claim 1, wherein the formulation is in the form of a solution, emulsion, microemulsion, lotion, gel, foam, milk, micellar water, oil, tensiolite, suspension, cream, balm, ointment, paste, cream-gel, stick, or wax.

10. The topical pharmaceutical formulation according to claim 1, wherein the formulation is in a gel or cream-gel form.

11. The topical pharmaceutical formulation according to claim 1, wherein the water-based composition is present in an amount between 0.5% and 20% by weight, relative to the total weight of the topical pharmaceutical formulation.

12. A method of treating a syndrome associated with chronic pelvic pain, the method comprising:
applying the topical pharmaceutical formulation of claim 1,
wherein the syndrome associated with chronic pelvic pain is at least one selected from the group consisting of vulvodynia, vestibulodynia, chronic prostatitis and prostatodynia, abacterial cystitis, painful bladder syndrome, urethral syndrome, dyspareunia, anus elevator syndrome, myofascial pain, coccicodynik coccigodynia, proctalgia fugax, and pudendal neuralgia.

13. A method of treating a syndrome associated with chronic pelvic pain, the method comprising:
applying topically the topical pharmaceutical formulation of claim 1 with a dynamic electroporation apparatus.
